SEQUENCE LISTING (6 sequences)

SEQ ID NO: 1 - DNA, 27 bp, Artificial Sequence
  Tau F-primer
ggggtaccccatggctgagccccgcca

SEQ ID NO: 2 - DNA, 32 bp, Artificial Sequence
  Tau R-primer
ccgctcgagcttattacaaaccctgcttggcc

SEQ ID NO: 3 - DNA, 32 bp, Artificial Sequence
  HT-Tau F-primer
ggggtaccccgcagaaatcggtactggctttc

SEQ ID NO: 4 - DNA, 30 bp, Artificial Sequence
  HT-Tau R-primer
ggggtaccggatccagtcgactgaattcgc

SEQ ID NO: 5 - DNA, 25 bp, Artificial Sequence
  C-terminal truncated Tau F-primer
gctgagccccgccaggagttcgaag

SEQ ID NO: 6 - DNA, 32 bp, Artificial Sequence
  C-terminal truncated Tau R-primer
taattaagcctcgagtcattcaatctttttat

(19) United States
(12) Patent Application Publication
(10) Pub. No.: US 2021/0341448 A1
(43) Pub. Date: Nov. 4, 2021

(54) SCREENING METHOD FOR THERAPEUTIC DRUG OR PROPHYLACTIC DRUG FOR TAUOPATHY AND DIAGNOSTIC METHOD FOR TAUOPATHY

(71) Applicant: National Center for Geriatrics and Gerontology, Aichi (JP)

(72) Inventors: Akiyoshi KAWAI, Aichi (JP); Tetsuya KIMURA, Aichi (JP); Hasi HUHE, Aichi (JP)

(21) Appl. No.: 17/284,220

(22) PCT Filed: Oct. 31, 2019
(86) PCT No.: PCT/JP2019/042743

§ 371 Date: Apr. 9, 2021

(30) Foreign Application Priority Data
Nov. 1, 2018 (JP) 2018-206594

(51) Int. Cl. G01N 33/15; C07K 14/705; G01N 33/543; G01N 33/68

(52) U.S. Cl. CPC G01N 33/15; C07K 14/70571; G01N 2333/70571; G01N 33/6896; G01N 33/5436

(57) ABSTRACT

Provided is a screening method for an agent for treating or preventing tauopathy, the method comprising (1) a step of contacting an NMDA-type glutamate receptor with a tau oligomer in the presence or absence of a candidate compound, and (2) a step of evaluating a direct binding of the tau oligomer to the NMDA-type glutamate receptor. Further provided is a test method for tauopathy, the method comprising (1) a step of contacting an NMDA-type glutamate receptor with a sample isolated from a subject, and (2) a step of quantifying tau oligomers directly binding to the NMDA-type glutamate receptor.

### RELATED APPLICATIONS

This application is a 35 U.S.C. § 371 national phase application of PCT Application PCT/JP2019/042743, filed Oct. 31, 2019, which claims priority to Japanese Application No. 2018-206594, filed Nov. 1, 2018. The entire contents of each are incorporated herein by reference in its entirety.

### STATEMENT REGARDING ELECTRONIC FILING OF A SEQUENCE LISTING

A Sequence Listing in ASCII text format, submitted under 37 C.F.R. § 1.821, entitled 5576-379_ST25.txt, 1,566 bytes in size, generated on Apr. 6, 2021, and filed via EFS-Web, is provided in lieu of a paper copy. This Sequence Listing is hereby incorporated by reference into the specification for its disclosures.

### TECHNICAL FIELD

The present invention relates to a screening method for an agent for treating or preventing tauopathy and to a diagnostic method for tauopathy.

### BACKGROUND ART

Currently, the number of patients with dementia has been increasing with the aging of the population, and establishment of therapeutic methods therefor is considered to be an urgent task. Dementia refers to a continuous disabling condition in daily life and social life caused by an irreversibly decline, for some acquired reasons, of higher brain functions that had once normally developed. Most dementia is neurodegenerative diseases such as Alzheimer's disease (AD) and frontotemporal lobar degeneration (FTLD). Neurofibrillary tangle (NFT) is known as a consistent characteristic having a high correlation with the degree of disability in cognitive function by the neurodegenerative diseases (Non-Patent Document 1). The major structural element of NFT is a tau protein, which is one of the microtubule-associated proteins (MAP), and tau proteins lose the microtubule binding ability when hyperphosphorylated, and aggregate to form NFT.

The NFT formation is considered deeply associated with dementia symptoms. As a matter of fact, it has been confirmed by the analysis of postmortem brains of AD patients that cognitive ability of patients has an inverse correlation with the frequency of NFT in the cerebral cortex and the hippocampus (Non-Patent Document 2) and also has an inverse correlation with the synaptic density (Non-Patent Document 3). On the other hand, it has been revealed by an experiment using model animals in which tau proteins are conditionally overexpressed that NFT itself has no strong synaptic toxicity or neurotoxicity such as synaptic depression/loss or induction of neuron death (Non-Patent Document 4). These findings lead to the concept that neurotoxicity is expressed in the process of the NFT formation.

Various treatments for tauopathy by inhibiting the formation of NFT have been attempted (Non-Patent Document 5) and these are roughly classified into (1) inhibition of phosphorylation of tau proteins, (2) inhibition of polymerization of tau proteins, and (3) tau immunotherapy. The approach of (1) proposes a use of an inhibitor against kinases such as glycogen synthase kinase 3β (GSK3β). However, as the kinases involved in the phosphorylation of tau proteins are also involved in the phosphorylation of various proteins other than the tau proteins, the continuous inhibition of them poses side effects, which is a problem. In the approach of (2), compounds modifying a disulfide (S—S) bond such as methylene blue is used as a polymerization inhibitor of tau proteins. However, as the S—S bond determines the secondary structure of various proteins, side effects are likely to be caused similarly. Furthermore, the compound modifying an S—S bond has the redox activity and thus generates radicals, thereby likely being toxic. In the approach of (3), immunization is carried out using a synthesized phosphorylated tau protein to thereby generate an antibody to inhibit the formation of NFT. However, the effect of tau immunotherapy has been confirmed only in transgenic mice which express a mutant tau protein. Additionally, the antibody has a low migration rate through the blood brain barrier (about 0.1 to 0.2%), and sufficient clinical effects are less likely to be obtained, which is a problem. Furthermore, the tau protein contains a large number of phosphorylation sites (Non-Patent Document 6), and it is not understood at present at which site the phosphorylation needs to be targeted for effective tauopathy treatment.

Recently, a report has been made that amounts of phosphorylated tau proteins and tau oligomers in the human cerebrospinal fluid (CSF) increase as AD pathological conditions advance (Non-Patent Document 7). Additional report has also been made that, in animal experiments, extracellular tau oligomers inhibit the formation of long-term potentiation (LTP), which is considered the mechanism of memory and learning (Non-Patent Document 8). These studies suggest that the tau oligomer has something to do with synaptic toxicity and neurotoxicity, but the mechanism by which the tau oligomer is involved in the development of neurotoxicity is still not understood.

### CITATION LIST

### Non-Patent Document

[Non-Patent Document 1] Burns A, O'Brien J, Ames D Edition, 2005, Dementia 3rd Edition, pp. 408-464
[Non-Patent Document 2] Nelson P T, Break H, Markbery W R, (2009), Journal of Neuropathology & Experimental Neurology, Vol. 68, pp. 1-14
[Non-Patent Document 3] Callahan L M, Coleman P D, (1995), Neurobiology of Aging, Vol. 16, pp. 311-314
[Non-Patent Document 4] Santacruz K, Lewis J, Spires T, et al., Science, (2005), Vol. 309, pp. 476-481
[Non-Patent Document 5] Noble W, Pooler A M, Hanger D P, Expert Opinion on Drug Discovery, (2011), Vol. 6, pp. 797-810
[Non-Patent Document 6] Sundermann F, Fernandez M P, Morgan R O, BMC Genomics, (2016), Vol. 17, p. 264
[Non-Patent Document 7] Hu Y Y, He S S, Wang X, et al., American Journal of Pathology, (2002), Vol. 160, pp. 1269-1278
[Non-Patent Document 8] Fa M, Puzzo D, Piacentini R, et al., Scientific Reports, (2016), Vol. 6, 19393

### SUMMARY OF INVENTION

### Technical Problem

On the other hand, the main symptom of dementia is defects of memory. In brain physiology, memory is associated with synaptic plasticity. Synaptic plasticity comes in a wide variety, but spike timing dependent plasticity (STDP) is considered important synaptic plasticity associated with memory and cognition (Song S, Miller K D, Abbott L F, Nature Neuroscience, (2000), Vol. 3, pp. 919-926). STDP is induced depending on the timings of excitement of a presynaptic cell and excitement of a postsynaptic cell, and it has been revealed that the NMDA-type glutamate receptor is deeply involved therein (Caporale N, Dan Y, Annual Reviews in Neuroscience, (2008), Vol. 31, pp. 25-46).

The present invention has an object to reveal the relationships of the tau oligomer with the expression mechanism of synaptic toxicity, and then provide a therapeutic drug for dementia that has not existed heretofore and a highly accurate diagnostic method.

### Solution to Problem

The present inventors have earnestly researched, and as a result, found that a tau oligomer binds to an NMDA-type glutamate receptor and directly stimulates the NMDA-type glutamate receptor, thereby causing synaptic toxicity.

Specifically, according to an embodiment, the present invention provides a screening method for an agent for treating or preventing tauopathy comprising: (1) a step of contacting an NMDA-type glutamate receptor with a tau oligomer in the presence or absence of a candidate compound, and (2) a step of evaluating a direct binding of the tau oligomer to the NMDA-type glutamate receptor.

Additionally, the present invention provides, according to an embodiment, a test method for tauopathy comprising: (1) a step of contacting an NMDA-type glutamate receptor with a sample isolated from a subject, and (2) a step of quantifying tau oligomers directly binding to the NMDA-type glutamate receptor.

The NMDA-type glutamate receptor is preferably contained on a cell membrane or a liposomal membrane while maintaining a physiological function.

The NMDA-type glutamate receptor is preferably isolated from a cell membrane or a liposomal membrane while maintaining a quaternary structure.

Alternatively, the NMDA-type glutamate receptor is preferably contained on a cell membrane or a liposomal membrane while maintaining a physiological function.

The tau oligomer or the NMDA-type glutamate receptor is preferably immobilized on a solid support.

The step (2) is preferably carried out by ELISA, a protein array, or a surface plasmon resonance analysis.

The method preferably further comprises (3) a step of measuring a calcium influx through the NMDA-type glutamate receptor into a cell or a liposome.

The method preferably further comprises (4) a step of measuring incorporation of a membrane protein into a cell or a liposome.

The tau oligomer preferably consists of 2 to 40 tau proteins, and more preferably consists of 3 to 20 tau proteins.

The tau oligomer preferably comprises, as a structural component, a tau protein comprising a phosphorylated amino acid in the C-terminal region downstream of an amino acid corresponding to the 373 position numbered according to the 2N4R isoform.

The tau oligomer preferably comprises, as a structural component, a tau protein in which serine corresponding to the 409, 412, 413 and/or 416 position numbered according to the 2N4R isoform is phosphorylated.

The tauopathy is preferably Alzheimer's disease, corticobasal degeneration, progressive supranuclear palsy, Pick's disease, argyrophilic grain dementia, multiple system tauopathy with presenile dementia (MSTD), frontotemporal dementia with parkinsonism linked to chromosome 17 (FTDP-17), dementia with neurofibrillary tangles, diffuse neurofibrillary tangles with calcification (DNTC), white matter tauopathy with globular glial inclusions (WMT-GGI), or frontotemporal lobar degeneration with tau-positive inclusions (FTLD-tau).

### Advantageous Effects of Invention

According to the method of the present invention, compounds capable of reducing the synaptic toxicity caused when a tau oligomer directly binds to an NMDA-type glutamate receptor and stimulates the NMDA-type glutamate receptor can be obtained, and such a compound can be a novel therapeutic drug or a prophylactic drug for dementia, and hence useful.

Furthermore, the quantification of the tau oligomers directly binding to the NMDA-type glutamate receptors present in a sample isolated from a subject enables the detection of tauopathy with high accuracy.

### BRIEF DESCRIPTION OF DRAWINGS

FIG. 1 is a drawing showing reduced synapse transmission efficiency of an aging mouse brain slice specimen to which tau oligomers have been exposed.

FIG. 2 is a graph showing changes in amounts of the postsynaptic NMDAR and AMPAR in a brain slice specimen from an aging mouse to which tau oligomers have been exposed.

FIG. 3 is a graph showing amount changes amounts of the postsynaptic NMDAR and AMPAR in a brain slice specimen from an aging tau knockout mouse to which tau oligomers have been exposed.

FIG. 4 is a drawing showing results of coimmunoprecipitation of a tau and NMDAR using an anti-tau antibody on fractions of membrane proteins prepared from a brain slice specimen of an aging tau knockout mouse to which tau oligomers have been exposed.

FIG. 5 is a drawing showing calcium influx through NMDAR into NT2-N cells to which tau oligomers have been administered.

FIG. 6A to 6B are graphs showing quantification analysis results of calcium influx through NMDAR into the NT2-N cells to which tau oligomers have been administered.

FIG. 7 is a fluorescence microscope image confirming the adhesion of tau oligomers to the NT2-N cell surface.

FIG. 8 is a graph showing results of measurement of tau oligomers incorporation into the NT2-N cells.

FIG. 9 is a drawing showing results of pull-down assay using HT-tau oligomers and membrane protein fractions prepared from a brain slice specimen of a tau knockout mouse.

FIG. 10 is a drawing showing far-western blot results using HT-tau oligomers and NMDAR complex purified from a brain slice specimen of a tau knockout mouse.

FIG. 11A to 11C is drawings showing results of dot blot comparing the binding ability of gel-filtered-tau-oligomer fractions 6 to 10 to NMDAR.

FIG. 12 is a drawing showing results of blue-native PAGE/western blot of gel-filtered-tau-oligomer fractions 6 and 8.

FIG. 13 is a drawing showing a result of sandwich ELISA detecting and quantifying the binding of a membrane proteins and the tau oligomers.

FIG. 14 is a drawing showing a result of sandwich ELISA detecting and quantifying the binding of the NMDA receptor and the tau oligomer.

FIG. 15 is a drawing showing a result of sandwich ELISA detecting and quantifying the binding of the NMDA receptor and the tau oligomer in the presence or absence of conantokin-G.

FIG. 16 is a drawing showing a result of thioflavin T assay confirming the time course of the formation of tau oligomers.

FIG. 17 is a drawing showing a result of sandwich ELISA detecting and quantifying the binding of the tau oligomer reconstituted from tau monomers and a membrane protein.

### DESCRIPTION OF EMBODIMENTS

Hereinafter, the present invention will be described in detail but is not limited to the embodiments described herein.

According to the first embodiment, the present invention is a screening method for an agent for treating or preventing tauopathy, the method comprising (1) a step of contacting an NMDA-type glutamate receptor with a tau oligomer in the presence or absence of a candidate compound, and (2) a step of evaluating a direct binding of the tau oligomer to the NMDA-type glutamate receptor.

The “tauopathy” is a general term for, among neurodegenerative diseases, those in which abnormal accumulation of tau proteins aggregated in neurons is characteristically observed. Examples of the tauopathy include, but not limited to, Alzheimer's disease, corticobasal degeneration, progressive supranuclear palsy, Pick's disease, argyrophilic grain dementia, multiple system tauopathy with presenile dementia (MSTD), frontotemporal dementia with parkinsonism linked to chromosome 17 (FTDP-17), dementia with neurofibrillary tangles, diffuse neurofibrillary tangles with calcification (DNTC), white matter tauopathy with globular glial inclusions (WMT-GGI), and frontotemporal lobar degeneration with tau-positive inclusions (FTLD-tau).

In the present embodiment, “treating” means to block or alleviate, in animals affected with tauopathy, the advancement or aggravation of pathological conditions of such a patient, and not only includes complete recovery from the disease, but also alleviation of various symptoms of the disease. The “preventing” means to prevent an animal likely to be affected with tauopathy from being affected therewith.

In the screening method of the present embodiment, tau oligomers are contacted with NMDA-type glutamate receptors in the presence or absence of a candidate compound.

In the present embodiment, the “tau oligomer” means a polymer of two or more tau proteins (monomers). The tau oligomer according to the present embodiment preferably consists of 2 to 40 tau proteins, and particularly preferably 3 to 20 tau proteins. The tau oligomer according to the present embodiment does not include tau fibers formed by binding tau oligomers each other.

The “tau protein” composing the tau oligomer according to the present embodiment (also simply referred to as “tau” herein) includes six splice variants of wild-type tau proteins expressed from human tau genes, specifically, the 0N3R isoform, the 1N3R isoform, the 2N3R isoform, the 0N4R isoform, the 1N4R isoform, and the 2N4R isoform, and also mutants and homologues thereof as long as equal physiological functions are maintained therein. Furthermore, tau proteins composing the tau oligomer according to the present embodiment can be phosphorylated or not phosphorylated.

The tau oligomer according to the present embodiment can include one or more kinds of tau proteins selected from the above phosphorylated or non-phosphorylated tau proteins. The tau oligomer according to the present embodiment preferably contains a phosphorylated tau protein as the structural component. The ratio of phosphorylated tau proteins contained in the tau oligomer according to the present embodiment is preferably 80% or more, and particularly preferably 90% or more. Additionally, the amino acid to be phosphorylated in a tau protein can be an amino acid at any position, but is preferably one or more amino acids contained in the C-terminal region downstream of an amino acid corresponding to the 373 position numbered according to the 2N4R isoform, and particularly preferably serine corresponding to the 409, 412, 413 and/or 416 position numbered according to the 2N4R isoform.

The tau oligomer according to the present embodiment can be prepared by polymerizing tau proteins (monomers). The tau protein can be prepared by biosynthesis, for example, by introducing an expression vector containing DNA encoding a tau protein into a host cell to express the tau protein. The host cells usable for expressing a tau protein include, for example, bacteria, yeasts, and mammalian cells, and preferably E. coli such as BL21 and Rosetta can be used. For the expression vector, a suitable expression vector can be selected and used in accordance with the kind of a host cell to be used. For example, when E. coli is used as the host cell, an E. coli expression plasmid such as pT7 vector and pET vector can be used, and when a mammalian cell is used as the host cell, an animal cell expression plasmid such as pcDNA3.1 or a virus vector such as a retrovirus and an adenovirus can be used. The introduction of an expression vector into a host cell can be carried out by a well-known method suitable for the host cell such as electroporation and lipofection. The tau protein expressed can be purified by a conventionally known technique such as column chromatography. Furthermore, the tau proteins composing a tau oligomer according to the present embodiment can be those to which a tag such as 6× His, HA, Myc, FLAG, or HaloTag or a marker protein such as GFP is added at the N-terminal and/or the C-terminal, and in such an instance, a tau protein can be affinity purified using an antibody to the tag or the marker protein.

A tau oligomer can be prepared by adding tau proteins to a brain tissue extract and polymerizing them. The animal from which brain tissue is derived can be any mammal such as a mouse, a rat, a rabbit, a dog, a non-human primate, a human, and is preferably a human. The brain tissue extract can be prepared by a conventionally known method such as a method involving physically disrupting brain tissues or a method involving dissolving brain tissues using a surfactant such as CHAPS. Alternatively, the tau oligomer can also be prepared by adding tau proteins to an artificial reaction solution prepared according to the composition of brain tissue extract and polymerizing them. The artificial reaction solution can be prepared by, for example, mixing suitable components, preferably a kinase such as GSK3β, MAP kinase, or CAM kinase, a phosphate donor such as ATP or GTP, and the like, with a buffer solution such as 2 to 50 mM of HEPES (pH 7.0 to 8.0) or 2 to 50 mM of Tris (pH 7.0 to 8.0) and/or a salt (for example, 100 to 500 mM of NaCl, 0 to 4 mM of KCl, or 0 to 6 mM of MgCl₂). The polymerization reaction can be carried out by incubating a brain tissue extract or an artificial reaction solution to which tau proteins are added for a certain period of time, and for example, by incubating it 1 to 3 days at 37° C.

The “NMDA-type glutamate receptor” (also referred to as “NMDA receptor” or “NMDAR” herein) is a cation permeable ion-channel-coupled receptor mainly present on the membrane of presynaptic and postsynaptic cells of the central neuronal system, and plays an important role in the various neural activities such as synaptic plasticity to begin with. Known subunits composing the NMDA receptor are NR1, NR2a, NR2b, NR2c, NR2d, NR3a, and NR3b, and the NMDA receptor according to the present embodiment can be those including one or more kinds of subunits selected from the above. The NMDA receptor according to the present embodiment is preferably a heterotetramer including NR1 and a single or several kinds of NR2.

The NMDA receptor according to the present embodiment can be prepared from cells expressing the NMDA receptor. The cell expressing the NMDA receptor can be, for example, brain tissue sections or neurons isolated from a mammal, neuronal cell lines such as NT2-N, neurons differentiated from iPS cells, and cell lines, such as HEK293, HeLa, CHO, and COS7, expressing the NMDA receptor by introducing an expression vector containing DNA encoding the NMDA receptor. The NMDA receptor can be prepared in accordance with a general membrane receptor purification method and, for example, the above disrupted cell solution can be ultracentrifuged in a sucrose density gradient to thereby separate and collect membrane vesicle fractions containing NMDA receptors. This enables the purification of the NMDA receptor as being contained on the liposomal membrane while maintaining a physiological function. “Maintaining a physiological function” as used for the NMDA receptor herein refers to the NMDA receptor maintaining a neurotransmitter-dependent and/or an electric potential-dependent ion channel activity. Preferably, the membrane vesicle fraction containing the NMDA receptor is synaptosome and/or synaptoneurosome fraction.

Furthermore, the NMDA receptor according to the present embodiment can be isolated from the cell membrane or the liposomal membrane while the NMDA receptor maintains the quaternary structure. “Maintaining the quaternary structure” as used for the NMDA receptor herein refers to the NMDA receptor maintaining the whole structure composed of subunits and optionally maintaining the binding to scaffolding proteins such as PSD95. The NMDA receptor can be, for example, solubilized by dissolving the cell membrane or the liposomal membrane using a surfactant such as 1% sodium cholate, 0.38% sodium deoxycholate, or 1% n-dodecyl-β-D-maltoside (DDM), and thus, can be separated while maintaining the quaternary structure.

The contact of the tau oligomer and the NMDA receptor can be carried out by allowing both of them to be present in a buffer solution such as HEPES-buffered artificial cerebrospinal fluid with or without addition of a candidate compound and incubating for a certain period of time. The candidate compound is not particularly limited and examples include proteins, peptides, nucleic acids, non-peptide compounds, synthetic compounds, cell extracts, plant extracts, and animal tissue extracts, which can be novel substances or known substances. The concentration of the candidate compound varies depending on the kind of the compound and can be suitably selected from a range of, for example, 0.01 nM to 100 μM. The concentrations and the incubation time of the tau oligomer and the NMDA receptor can be suitably determined in accordance with a binding analysis technique to be employed. For example, the concentration of the tau oligomer can be in a range from 0.01 to 1 nM, the concentration of the NMDA receptor can be in a range from 0.01 to 1 nM, and the incubation time can be in a range from 10 minutes to 5 hours. The above concentrations of the tau oligomers and the NMDA receptors are defined in the condition that the respective quaternary structure is considered to be one molecule.

Subsequently, the direct binding of the tau oligomer to the NMDA receptor is evaluated. The binding of the tau oligomer to the NMDA receptor can be evaluated by any technique for analyzing a protein-protein interaction and examples of such a technique include, but not limited to, coimmunoprecipitation, pull-down assay, ELISA, protein array, surface plasmon resonance analysis (SPR), and fluorescence resonance energy transfer (FRET). The binding analysis technique by the method of the present embodiment is preferably ELISA, protein array, or SPR, and can be carried out using an analysis system suitable for each of them.

Either or both of the tau oligomer and the NMDA receptor herein can be immobilized on a solid support. The solid support can be, for example, those having as the main component a semiconductor such as silicon, an inorganic substance such as glass, a polymeric substance such as polystyrene, polyethylene terephthalate, or PVDF, and can be suitably selected according to the binding analysis technique. Furthermore, the shape of solid support can be any shape suitable for the use in the analysis system such as a membrane, a bead, a glass slide, a multi-well plate, a microtiter plate, a multiarray chip, and a sensor chip. The immobilization of the tau oligomer or the NMDA receptor on the solid support can be carried out by an already established general ligand immobilization method. For example, they can be directly coupled on the surface of the solid support by covalent binding, or biotinylated tau oligomer or NMDA receptor can be indirectly coupled on the solid support coated with streptavidin. For immobilizing the NMDA receptor on the surface of the solid support, when the NMDA receptor is purified on a liposomal membrane while maintaining a physiological function, the liposome can be immobilized on the solid support, and when the NMDA receptor is isolated from a lipid membrane while maintaining the quaternary structure, the NMDA receptor itself can be immobilized on the solid support directly or through an anti-NMDA receptor antibody.

The tau oligomer or the NMDA receptor is preferably labelled with a fluorescent dye for detecting the binding. The kind of a fluorescent dye is not particularly limited, and for example, fluorescein and derivatives thereof, rhodamine and derivatives thereof, carbocyanine dye, indocyanine green dye, phthalocyanine dye, squarylium dye, BODIPY, Cy5.5, and dansyl can be used. The labelling method of a protein is already established and, for example, a label is introduced to an amino group of the tau oligomer or the NMDA receptor. For fluorescence-labelling the NMDA receptor purified on a liposomal membrane while maintaining a physiological function, either the membrane of or inside the liposome can be labelled with fluorescence but the labelling is preferably achieved by enclosing a fluorescent dye inside the liposome.

In the screening method of the present embodiment, when the direct binding of the tau oligomer to the NMDA receptor in the presence of a candidate compound is significantly reduced in comparison with the binding in absence of the candidate compound, it can be determined that such a candidate compound is a potential compound as an agent for treating or preventing tauopathy. On the other hand, when the direct binding of the tau oligomer to the NMDA receptor in the presence of a candidate compound is equal to or increased more than the binding in absence of the candidate compound, it can be determined that such a candidate compound is not a potential compound as an agent for treating or preventing tauopathy.

The screening method of the present embodiment can further comprise (3) a step of measuring a calcium influx through the NMDA receptor into a cell or a liposome. The calcium influx into a cell or a liposome can be measured by, for example, loading a fluorescent calcium indicator in a cell or a liposome thereby detecting calcium concentration changes in the cell or the liposome. The fluorescent calcium indicator can be one-excitation wavelength/one-emission wavelength fluorescent indicator such as Fluo-3, Fluo-4, and Indo-1, or two-excitation wavelength/one-emission wavelength fluorescent indicator such as Fura-2. When the NMDA receptor is contained on a liposomal membrane while maintaining a physiological function, a use of a fluorescent calcium indicator having different excitation wavelength/fluorescence emission wavelength from the fluorescent dye for detecting the binding of the tau oligomer enables the simultaneous evaluations on the binding of the NMDA receptor to the tau oligomer and the calcium influx into a liposome through the MDA receptor. Alternatively, a ratiometric fluorescent dye such as Fura-2 can be used to measure fluorescence unaffected by calcium concentration changes (the fluorescence at 360 nm excitation for Fura-2) in addition to the fluorescence for measuring calcium concentration changes (the fluorescence at 340 nm and 380 nm excitations for Fura-2), whereby the binding of the NMDA receptor to the tau oligomer and the calcium influx into a liposome through the NMDA receptor can be simultaneously evaluated without using an additional fluorescent label.

In the screening method of the present embodiment, when the calcium influx through the NMDA receptor in the presence of a candidate compound is significantly reduced in comparison with the calcium influx in the absence of the candidate compound, it can be determined that such a candidate compound is a potential compound as an agent for treating or preventing tauopathy. On the other hand, when the calcium influx through the NMDA receptor in the presence of a candidate compound is equal to or increased more than the calcium influx in absence of the candidate compound, it can be determined that such a candidate compound is not a potential compound as an agent for treating or preventing tauopathy.

The screening method of the present embodiment can further comprise (4) a step of measuring incorporation of a membrane protein into a cell or a liposome. The incorporation of a membrane protein can be measured by, for example, labelling the membrane protein with a pH-responsive fluorescent probe and detecting pH changes around the membrane protein that has been incorporated. The pH-responsive fluorescent probe can be a fluorescent dye such as AcidiFluor™ ORANGE, or a fluorescent protein such as pHluorin or pHluorin2. The membrane protein to be labelled with a pH-responsive fluorescent probe is not particularly limited and can be, for example, the NMDA receptor and AMPA-type glutamate receptor (AMPA receptor). The pH-responsive fluorescent probe can be added to a membrane protein by a known chemical technique or a genetic engineering technique. Alternatively, as the tau oligomer binds stably to the NMDA receptor, labelling the tau oligomer with a pH-responsive fluorescent probe enables indirect labelling of the NMDA receptor.

Alternatively, changes in the synapse transmission intensity can also be measured so as to measure the incorporation of a receptor protein involved in the synapse transmission such as NMDA receptor and AMPA receptor into a cell or a liposome. The synapse transmission intensity can be evaluated by, for example, the measurement of an extracellular potential by measuring a local field potential (LFP), the measurement of an intracellular potential by a patch-clamp method, or the measurement of membrane potential changes using a membrane potential-sensitive dye such as Di-3-ANEPPDHQ.

In the screening method of the present embodiment, when the incorporation of a membrane protein into a cell or a liposome is significantly reduced in comparison with the incorporation of a membrane protein in the absence of a candidate compound, it can be determined that such a candidate compound is a potential compound as an agent for treating or preventing tauopathy. On the other hand, when the incorporation of a membrane protein into a cell or a liposome in the presence of a candidate compound is equivalent to or increased more than the incorporation of a membrane protein in the absence of the candidate compound, it can be determined that such a candidate compound is not a potential compound as an agent for treating or preventing tauopathy.

The method of the present invention is useful for screening a candidate compound for a therapeutic drug or a prophylactic drug for dementia.

According to the second embodiment, the present invention is a test method for tauopathy, the method comprising (1) a step of contacting an NMDA-type glutamate receptor with a sample isolated from a subject, and (2) a step of quantifying tau oligomers directly binding to the NMDA-type glutamate receptor. The “NMDA-type glutamate receptor”, “tau oligomer”, and “tauopathy” according to the present embodiment are the same as defined in the first embodiment.

The “test” in the present embodiment means to numerically quantify the tau oligomers directly binding to the NMDA receptors as an indicator or detect the presence or absence thereof. Based on the test result, a doctor can determine/diagnose whether or not a subject is affected with tauopathy and can decide a suitable course of treatment.

The “subject” in the present embodiment is an individual animal that can be affected with tauopathy. The animal can include mammals such as a mouse, a rat, a rabbit, a dog, a non-human primate, a human, and is preferably a human.

The “sample” according to the present embodiment is a biological sample collectable from a subject and can be, for example, tissues, cells, or body fluids derived from a subject but not limited thereto. Preferable samples according to the present embodiment can be, for example, brain tissues and cerebrospinal fluid (CSF), with CSF being particularly preferable. The sample can be obtained from a subject by a method well known to those skilled in the art.

In the present embodiment, a sample isolated from a subject is contacted with the NMDA receptor. The contact of a sample and the NMDA receptor can be carried out by, for example, allowing both of them to be present in a buffer solution such as HEPES-buffered artificial cerebrospinal fluid and incubating for a certain period of time. The concentrations and the incubation time of the sample and NMDA receptors can be the same as defined in the first embodiment and can be suitably determined in accordance with a binding analysis technique to be employed.

Subsequently, the tau oligomer directly binding to the NMDA receptor is quantified. The technique for quantifying the binding of the tau oligomer and the NMDA receptor is the same as the evaluation technique for the binding of the tau oligomer and the NMDA receptor defined in the embodiment of the first embodiment.

The test method for tauopathy of the present embodiment can further comprise (3) a step of measuring a calcium influx through the NMDA receptor into a cell or a liposome, and/or (4) a step of measuring incorporation of a membrane protein into a cell or a liposome. These measurement procedures can be the same as defined in the first embodiment.

The test method for tauopathy of the present embodiment can further include a step of comparing the above quantitative results with a tau oligomer profile predetermined on a sample derived from a control who is not affected with tauopathy (a normal control sample). When the tau oligomer in a sample derived from a subject shows a significant increase than the normal value according to this comparison result, it can be determined that the subject may be affected with tauopathy. Specifically, in the test method for tauopathy of the present embodiment, when the direct binding of the tau oligomer to the NMDA receptor, the calcium influx through the NMDA receptor, and/or the incorporation of a membrane protein into a cell or a liposome show a significant increase above the normal value, it can be determined that a subject may be affected with tauopathy. In that sense, the test method for tauopathy according to the present embodiment can be a method for evaluating and determining whether or not a subject is affected with tauopathy, i.e., a diagnostic method. Additionally, comparing by the method according to the present embodiment the amounts of tau oligomer contained in samples collected from the same person before and after administrating a tauopathy therapeutic drug also enables the evaluation of the treatment effect.

The test method for tauopathy of the present embodiment enables the detection of tauopathy with high accuracy and is extremely useful.

### EXAMPLES

Hereinafter, the present invention will be further described with reference to examples. These are not intended to limit the present invention in any way.

### <1. Preparation of Tau Oligomer>

### (1-1) Preparation of Tau Monomer

The human tau 2N4R isoform monomer (hereinafter referred to as “tau monomer”) was prepared by the following procedure. The following set of primers was used with the tau/pET29b plasmid (Addgene, #16316) which contains a coding sequence of the human tau 2N4R isoform as a template, thereby to prepare a DNA fragment encoding the tau gene.

[Formula 1]
Forward primer:
(SEQ ID NO: 1)
GGGGTACCCCATGGCTGAGCCCCGCCA
Reverse primer:
(SEQ ID NO: 2)
CCGCTCGAGCTTATTACAAACCCTGCTTGGCC

The obtained DNA fragment was inserted to the XhoI/KpnI restriction enzyme sites of pET47b(+) (Novagen, #71461-3), which is an expression vector containing a sequence encoding a His×6 tag, thereby to obtain His6-tau monomer expression vector pET47b-His6-Tau. E. coli BL21(DE3) was transformed by pET47b-H6-Tau and a kanamycin resistant strain was obtained. The obtained strain was precultured in kanamycin-containing LB medium, 0.6 mM of IPTG was added after reaching OD600=0.6 to 1.0 to induce the expression of the protein, and was further cultured for 2 hours. The culture solution was centrifuged at 4° C., 6000×g, and the bacterial cells were collected and were stored −80° C. until purification.

The bacterial cells were suspended on ice in a binding buffer (20 mM of sodium phosphate, 500 mM of NaCl, 20 mM of imidazole, pH 7.4) containing 1% Protease Inhibitor Cocktail (Sigma-Aldrich), 0.2 mg/ml of lysozyme (Wako Pure Chemical Industries), 1 μM of PMSF (Nacalai Tesque Inc.), 0.1% of Triton X-100 (Nacalai Tesque Inc.) and 0.5 mM of DTT, disrupted by ultrasonic treatment, then centrifuged at 4° C., 20,000×g for 30 minutes thereby to collect the supernatant. The supernatant was filtered using a 0.2 μm filter and then applied to HisTrap HP column (GE Healthcare) equilibrated in advance with the biding buffer. Subsequently, an elution buffer (20 mM of sodium phosphate, 500 mM of NaCl, and 400 mM of imidazole) was applied to the column to elute the adsorbed substance. The obtained eluate was dialyzed to remove the imidazole and HEPES buffer (50 mM of HEPES, pH 7.4) was replaced. Then, the His-tag was cleaved by an HRV 3C protease (Takara Bio Inc) thereby to obtain a crude purified solution of the tau monomer.

The obtained crude purified solution was applied to HisTrap SP column (GE Healthcare), and then the adsorbed substance was eluted in a 0 to 1 M NaCl concentration gradient and a fraction eluted near 300 mM was collected. The purity of the tau monomer in the obtained fraction was confirmed by western blot and the CBB staining, then desalination by dialysis and concentration using an Amicon Ultra 10K centrifugal filter (Merck Millipore), and the buffer replacement by a HEPES buffer (40 mM of HEPES, pH 7.2) were carried out thereby to obtain a tau monomer sample.

### (1-2) Preparation of HaloTag-Fused Tau Monomer

Human tau 2N4R isomer monomer to which HaloTag was added at the N-terminal (hereinafter referred to as “HT-tau monomer”) was prepared by the following procedure. The following primer set was used with the pENTR4-HaloTag plasmid (Addgene, #29644) which contains a coding sequence of HaloTag as a template thereby to prepare a DNA fragment encoding the HaloTag.

[Formula 2]
Forward primer:
(SEQ ID NO: 3)
GGGGTACCCCGCAGAAATCGGTACTGGCTTTC
Reverse primer:
(SEQ ID NO: 4)
GGGGTACCGGATCCAGTCGACTGAATTCGC

The obtained DNA fragment was inserted to the Acc65 restriction enzyme site of pET47b-His6-Tau created in the above (1-1) to thereby obtain an His6-HT-tau monomer expression vector, pET47b-His6-HT-tau. The expression and purification of the protein were carried out by the same procedure as in the above (1-1) to obtain an HT-tau monomer sample, except that pET47b-His6-HT-tau was used instead of pET47b-H6-Tau.

### (1-3) Preparation of Tau Oligomers

A tau oligomer sample was prepared by the following procedure using the tau monomer sample prepared in the above (1-1). Brains were isolated from 3-week-old C57BL/6 mice and homogenized by adding 5-fold weight of lysis buffer (50 mM of Tris-HCl, 5 mM of EGTA, 2 mM of DTT, 50 mM of NaF, 1 mM of Na₃VO₄, 1% Protease Inhibitor Cocktail, and 1 μM of PMSF). The obtained homogenate was centrifuged at 4° C., 6000×g for 15 minutes to collect the supernatant, and the supernatant was ultracentrifuged at 400,000×g for 60 minutes to thereby obtain a mouse brain extract.

The tau monomer sample (final concentration 200 to 250 μg/ml) and the mouse brain extract (final concentration 50 to 100 μg/ml) were added to a reaction buffer (40 mM of HEPES, 3 mM of MgCl₂, 5 mM of EGTA, 2 mM of DTT, 2 μM of okadaic acid, 50 mM of NaF, 1 mM of Na₃VO₄, 1% Protease Inhibitor Cocktail, and 2 mM of ATP, pH 7.2) and incubated while mildly stirred at 37° C. 2 mM of ATP was added 12 to 24 hours later, and the reaction solution after 60 hours was centrifuged at 4° C., 400,000×g for 1 hour to thereby collect pellets. The obtained pellets were dissolved in 0.5 ml of HEPES-aCSF (10 mM of HEPES, 132 mM of NaCl, 2.5 mM of KCl, 1.3 mM of MgCl₂, 2.2 mM of CaCl₂, 10 mM of Glucose) to use as a tau oligomer sample. Tau oligomers were confirmed and quantified by blue-native PAGE and SDS-PAGE/western blot.

### (1-4) Preparation of HaloTag-Containing Tau Oligomer

A HaloTag-containing tau oligomer sample (hereinafter described as “HT-tau oligomer sample”) was prepared by the same procedure as in the above (1-3) except that a sample obtained by mixing the tau monomer sample and the HT-tau monomer sample prepared in the above (1-1) and (1-2) in a ratio of 4:1 was used instead of the tau monomer sample prepared in the above (1-1).

### <2. Identification of Physiological Activity of the Tau Oligomer Sample>

### (2-1) Creation of Brain Slice Specimen

Brain slice specimens from mice were prepared by the following procedure to evaluate impacts by the tau oligomer on synapse transmission efficiency. 20- to 23-month-old C57BL/6 mice (C57BL/6J or C57BL/6N, male) were cervically dislocated and decapitated to isolate the brain. The obtained brain was thoroughly cooled in aCSF (124 mM of NaCl, 3 mM of KCl, 26 mM of NaHCO₃, 1.25 mM of NaH₂PO₄, 2 mM of CaCl₂, 1 mM of MgSO₄, 10 mM of D-glucose) bubbled with a 95% O₂/5% CO₂ mixed gas, then dissected at the center of the cerebrum along the coronal plane, and further the dissected posterior site of the brain was sliced along the horizontal cross sections to a thickness of 350 μm using a vibratome (Leica, VT-1200S). The obtained sections were cut under a stereo microscope and were divided into regions containing the hippocampus/entorhinal cortex complex and the rest of them, and the regions containing the hippocampus/entorhinal cortex complex were used as the brain slice specimens.

### (2-2) Tau Oligomer Exposure to the Brain Slice Specimen

The obtained brain slice specimen was moved on a porous membrane filter (8 μm, Thermo Fisher Scientific, #140654) in aCSF to keep the brain slice specimen at the gas-liquid interface. At this time, aCSF was bubbled all the time using a 95% O₂/5% CO₂ mixed gas. 2 hours later, the tau oligomer sample (10 μg/ml in aCSF) prepared in the above (1-3) was exposed to the brain slice specimen. 2 to 3 hours later, the brain slice specimen was washed in fresh aCSF, then retained for 30 minutes or more and used for the following measurement. Additionally, brain slice specimens, prepared by the same procedure except that the tau oligomer sample was not exposed, were prepared as a negative control. The brain slice specimens exposed to the tau oligomer sample and the brain slice specimens not exposed to the tau oligomer sample were each prepared from 3 individual mice.

### (2-3) Measurement of Synapse Transmission Efficiency

The brain slice specimen was moved to a measurement chamber through which aCSF was perfused, and electric pulse stimulation of 1.5, 2, 3, or 4 nA was applied to record induced potential changes for the measurement of synapse strength from CA1 to CA3. A glass microelectrode having an electrode resistance of 500Ω was used for the recording electrode. The recorded waveform was analyzed using a self-made analysis software to calculate an amplitude of fiber-volley and an amplitude of excitatory postsynaptic potential (fEPSP) at each electric pulse stimulation.

The results are shown in FIG. 1. In the figure, the “pTauO” represents the results of brain slice specimens exposed to the tau oligomer sample and the “aCSF” represents the results of the negative controls. The fiber-volley reflects presynaptic membrane activities (that is, synapse input strength), and the fEPSP reflects postsynaptic membrane activities (that is, synapse output strength). The synapse transmission efficiency was evaluated by a fiber-volley/fEPSP ratio, and it is revealed that the synapse transmission efficiency of the brain slice specimens exposed to the tau oligomer sample was significantly reduced in comparison with the negative controls (p<0.0001, Extra sum-of-squares Test). These results showed that the tau oligomer induces long-term depression (LTD) of synapse.

### <3. Identification of Target Molecule of Tau Oligomer>

Subsequently, the following test was carried out to confirm whether or not the confirmed LTD induced by the tau oligomer in the above was NMDA receptor-dependent LTD (hereinafter described as “NMDAR-LTD”).

### (3-1) Changes in Amount of NMDA Receptors and AMPA Receptors on the Synapse Surface

As the molecule mechanism of NMDAR-LTD, it has been known that the AMPA receptors (hereinafter described as “AMPAR”) and/or the NMDA receptors (NMDAR) are incorporated into a cell by the endocytosis in the postsynaptic membrane caused by the activation of the NMDA receptors. Thus, an analysis was performed on the changes in amount of NMDARs and AMPARs on synapses in the brain slice specimens exposed to the tau oligomer sample and the brain slice specimens not exposed to the tau oligomer sample, prepared by the procedures of the above (2-1) and (2-2).

The brain slice specimens were frozen and disrupted in a 50-fold volume of homogenizing buffer (4 mM of HEPES, 2 mM of EGTA, 0.32 M of sucrose, pH 7.4) using a Teflon® homogenizer. The disrupted tissue solution was centrifuged at 4° C., 1,000×g for 15 minutes to thereby collect the supernatant, and the supernatant was further centrifuged at 4° C., 12,000×g for 15 minutes to thereby collect membrane fractions (pellets). The obtained membrane fractions were dissolved in tris-buffered saline containing 0.5% Triton X-100 (50 mM of Tris, 500 mM of NaCl, pH 7.4) and were centrifuged at 4° C., 20,000×g for 15 minutes. The supernatant was used as a 0.5% Triton X-100-soluble membrane fraction sample and the pellet was used as a 0.5% Triton X-100-insoluble membrane fraction sample. The western blot was carried out on the 0.5% Triton X-100-insoluble membrane fraction sample to quantify the AMPA receptor (GluA2 subunit) and the NMDA receptors (NR2a and NR2b subunits). The antibodies used were an anti-Glu2 antibody (Alomone Labs, #AGC-073) (1:1000 dilution), an anti-NR2A antibody (Alomone Labs, #AGC-002) (1:1000 dilution), and an anti-NR2B antibody (Alomone Labs, #AGC-003) (1:1000 dilution).

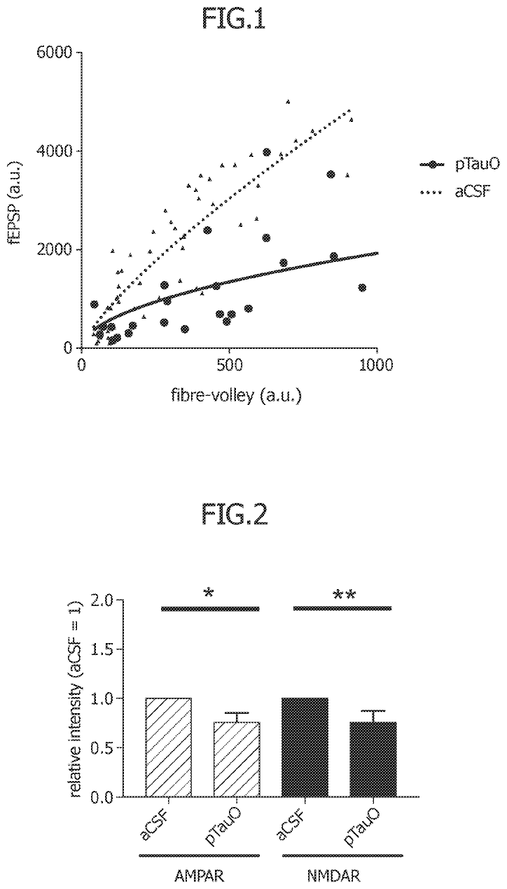

The results are shown in FIG. 2. In the figure, the “pTauO” shows the brain slice specimens exposed to the tau oligomer sample and the “aCSF” shows the negative controls, and the graph shows the normalized result when the receptor amount in aCSF was 1. The NMDARs and AMPARs were reduced on the postsynaptic site by the exposure of the tau oligomer sample (*: p<0.05, one sample t-test; **: p<0.01, one sample t-test; error bar: SEM). These results suggested that the LTD induced by the exposure of the tau oligomer sample was NMDAR-LTD.

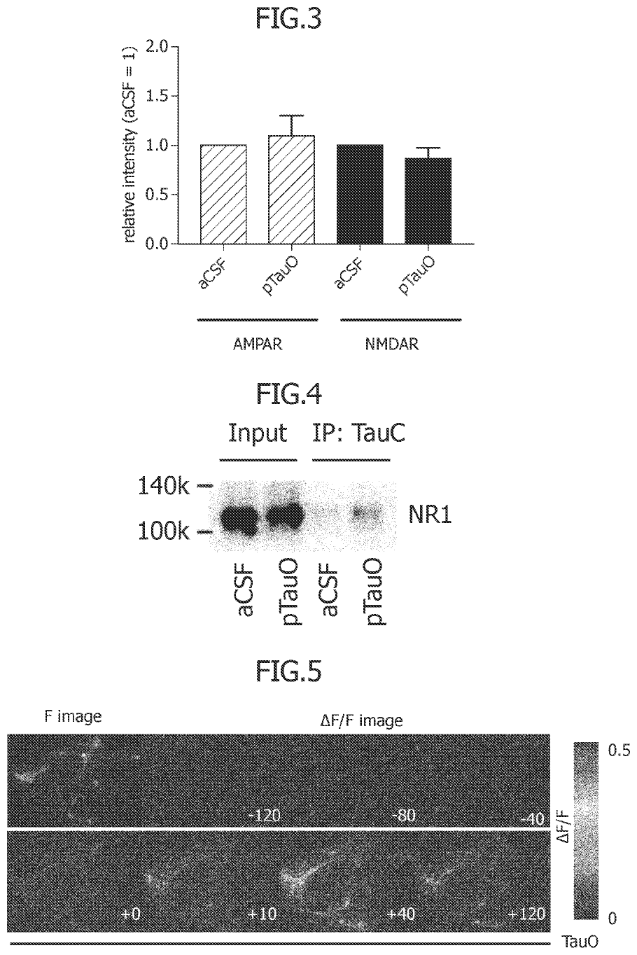

Furthermore, FIG. 3 shows the results of the brain slice specimens from tau knockout mice having disabled NMDAR-LTD (B6.129X1-Mapt^{tm1Hnd}/J, Jackson Laboratory) were prepared by the procedure of the above (2-1) and (2-2) and were analyzed similarly as above. In the tau knockout mice, the reduction of NMDARs and AMPARs on the synapse by the exposure of the tau oligomer sample was not observed. It was known that NMDAR-LTD depends on intracellular tau proteins, and the above results suggested that the LTD induced by the exposure of the tau oligomer sample was NMDAR-LTD.

### (3-2) Verification of Direct Interaction of Tau Oligomers and NMDAR

Whether or not the tau oligomers directly bind and interact with NMDARs was tested by the coimmunoprecipitation. The tau-knockout-brain slice specimens exposed to the tau oligomer sample prepared by the procedure of the above (2-1) and (2-2) was homogenized in 50-fold volume of the lysis buffer (4 mM of HEPES, 2 mM of EGTA, 0.32 M of sucrose, 1% Protease Inhibitor Cocktail, 1% Phosphatase Inhibitor Cocktail (Nacalai Tesque Inc., #07575-51)) and was centrifuged at 4° C., 12,000×g for 15 minutes to thereby collect pellets. The obtained pellets were dissolved in a 0.1% Triton X-100/TBS solution (25 mM of Tris-HCl, 150 mM of NaCl, 1 mM of EDTA, 1% NP-40, 5% glycerol, 0.1% Triton X-100, pH 7.4) and the resultant was used as a membrane protein solution. Coimmunoprecipitation on the obtained membrane protein solution was carried out by using an anti-tau antibody (TauC) (Dako, #A0024) (1:2000 dilution) and Pierce Direct Magnetic IP/Co-IP Kit (Thermo Fisher Scientific, #88828), in accordance with the recommended protocol by the kit. The collected coimmunoprecipitation samples were electrophoresed and then NMDAR was detected by the western blot. The antibody used was an anti-NR1 subunit antibody (Merck Millipore, #MAN363) (1:2000 dilution).

The results are shown in FIG. 4. In the figure, the “Input” represents the membrane protein solution before the coimmunoprecipitation (positive controls), and the “IP:TauC” represents the results of the coimmunoprecipitation samples by the anti-tau antibody (TauC). The NR1 subunit was not detected from the samples derived from the brain slice specimens not exposed to the tau oligomer sample, whereas a clear band of the NR1 subunits was detected in the samples derived from the brain slice specimens exposed to the tau oligomer sample. This result revealed that the tau oligomer directly binds to NMDAR.

### <4. Verification of NMDAR-Activation Ability of Tau Oligomer>

### (4-1) Preparation of Functional-NMDAR-Expressing Cell

Cultured neurons expressing NMDARs retaining physiological functions were prepared by the following procedure to evaluate the NMDAR activation ability of the tau oligomer. The NTERA-2 cl.D1[NT2/D1] cells (ATCC CRL-1973) (hereinafter described as “NT2 cell”), which are derived from a human pluripotent embryonic cancer, were adhesively cultured on 10-cm plastic dish in maintenance culture medium (high glucose DMEM (Sigma-Aldrich, D6429), 10% FBS, 100 U/ml penicillin/streptomycin) under the conditions of 37° C. and 5% CO₂ for 3 to 4 days until the confluency stage was attained. The NT2 cells were detached using Accutase (Innovative Cell Technologies, #AT104), were passaged, and were maintained over a period of 50 days. The medium was replaced 3 times a week.

The maintenance-cultured NT2 cells were dispersed in a low attachment petri dish (STAR SDish9015, RIKAKEN CO., LTD.) in a concentration of 1.5×10⁶ to 2.5×10⁶/dish, suspension-cultured under the conditions of 37° C. and 5% CO₂ on a shaker (100 rpm), and 1 day later were induced to differentiate by adding 1 μM of all-trans-retinoic acid (abcam), to thereby form spheroids. 14 days later, the spheroids were collected, seeded in a 10-cm dish coated with 5 μg/ml of poly-D-lysin (PDL) (Sigma-Aldrich)/Laminin (LAM) (iMatrix-511, Nippi Inc.) and adhesively cultured under the conditions of 37° C. and 5% CO₂. From the following day, the culture was carried out by adding 3 kinds of cell division inhibitors (10 μM of uridine, 10 μM of floxuridine, and 1 μM of AraC). Then, 3 days later, the cells were collected and reseeded on a 35-mm dish coated with PDL/LAM in a concentration of 0.1×10⁶/dish, and were cultured for 4 days in the presence of the 3 kinds of cell division inhibitors to thereby induce the differentiation to neurons.

The differentiation-induced cells were maintenance cultured in 0.2% NeuroCult™ SM1 Neuronal Supplement-containing BrainPhys™ Neuronal Medium (STEMCELL Technologies) under the conditions of 37° C. and 5% CO₂ for 2 to 3 months. The medium was replaced by half 3 times a week. Thus, neuron marker MAP2-positive cells were obtained and used as NT2-N cells. Furthermore, the expression of PSD-95 and GluN1 was confirmed from the membrane protein fractions prepared from the NT2-N cells (data not shown), thereby confirming that the NT2-N cell has the characteristic of central neurons and forms chemical synapses through glutamic acid.

### (4-2) Measurement of Calcium Influx Through NMDAR

A fluorescent calcium indicator Cal-520 AM (AAT Bioquest) or Fluo-8 AM (AAT Bioquest) was dissolved in 0.1 μM glycine-containing Mg-free HHBS (Hepes-buffered Hanks' balanced salt solution (1.26 mM of CaCl₂, 5.33 mM of KCl, 0.44 mM of KH₂PO₄, 4.17 mM of NaHCO₃, 137.93 mM of NaCl, 0.34 mM of Na₂HPO₄, 5.56 mM of D-glucose, 20 mM of HEPES, pH 7.4)) (4 μM and 2 μM, respectively). These solutions (1 ml) were loaded to the NT2-N cells under the conditions of 37° C. and 5% CO₂ (2 hours and 30 minutes, respectively). Subsequently, the cells were washed 3 times with HHBS and were placed in an incubator (37° C., 5% CO₂) of live cell Time Lapse Imaging System (BioStation IM-Q, Nikon Corporation) for 1 hour or more, and were used for the following measurement.

For analysis, 5 to 10 regions of the visual field of the NT2-N cells positioned in the system were manually set and the fluorescence intensities were recorded for each region, for Cal-520, every 1 minute for 1 hour continuously at excitation wavelength 480 nm/fluorescence emission wavelength 520 nm, and for Fluo-8, every 2 seconds for 5 minutes continuously at excitation wavelength 480 nm/fluorescence emission wavelength 520 nm. Also, 100 μl of the tau oligomer sample (0.03 μg/ml in HHBS), prepared in the above (1-3), was added after a certain period of time had passed since the records had begun (after 30 minutes for Cal-520, and after 120 seconds for Fluo-8).

The results of measurement by Fluo-8 are shown in FIG. 5. In the figure, the “F image” represents the fluorescence images, and the “ΔF/F image” represents changes in the fluorescence intensity. Additionally, the numeric characters represent the time at the image acquisition (second) when the time of administration of the tau oligomer sample was 0. After administration of the tau oligomer sample, the intracellular calcium concentration increased over time. On the other hand, the increase of the intracellular calcium concentration was not observed when the same amount of the reaction solution (tau not contained), used for preparing the tau oligomer sample in the above (1-3), was added.

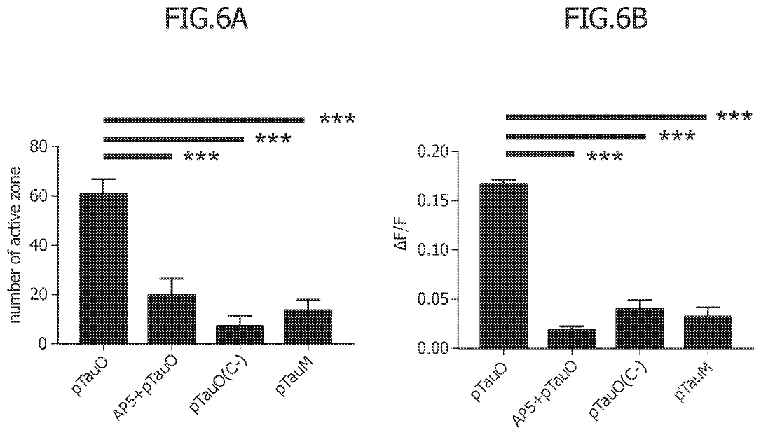

Furthermore, the results of measurement by Cal-520 are shown in FIG. 6A to 6B. In the figure, the “pTauO” represents the tau oligomer sample; the “APS+pTauO” represents the tau oligomer sample with 2-amino-5-phosphonopentanoic acid (AP5), an antagonist against NMDAR (50 μM); the “pTauO(C-)” represents a tau oligomer sample prepared by the same procedure as in the above (1-3), using tau monomers from which the C-terminal (the 373 amino acid and thereafter) was deleted; and the “pTauM” represents a phosphorylated tau monomer sample. FIG. 6A shows the number of zones in which a notable increase of calcium concentration (ΔF/F>0.4) was observed at the time of 3 minutes after the administration of the tau samples. FIG. 6B shows the changes in the fluorescence intensity of the above zones at the time of 3 minutes after the administration of the tau samples. The increase of the intracellular calcium concentration induced by the tau oligomer was lost by APS, thereby confirming that the tau oligomer has the NMDAR activation ability. Furthermore, the intracellular calcium concentration did not increase by the phosphorylated tau monomer or the oligomer consisting of the C-terminal truncated tau monomers, thereby revealing that the NMDAR activation ability of tau oligomers requires the C-terminal region downstream of the 373 amino acid of the tau.

The phosphorylated tau monomer was collected from the HT-tau oligomer sample prepared in the above (1-4) using Magne™ HaloTag beads (Promega, #G728A) (50 mg). Subsequently, the HaloTag was cleaved using TEV protease (Sigma-Aldrich, #T4455-10KU), and the tau monomers were detached from the beads. Then, the monomers were purified using Superdex 200 10/300 GL column (GE Healthcare) (the eluate: HEPES-aCSF; the flow rate: 0.5 ml/min) and were collected as a phosphorylated tau monomer sample. Also, the C-terminal truncated tau monomer was prepared by the same procedure as in the above (1-1) except that the following primer set was used.

[Formula 3]
Forward primer:
(SEQ ID NO: 5)
GCTGAGCCCCGCCAGGAGTTCGAAG
Reverse primer:
(SEQ ID NO: 6)
TAATTAAGCCTCGAGTCATTCAATCTTTTTAT

### <5. Evaluation on the Adhesion Activity of Tau Oligomers to Cell Surface>

### (5-1) Evaluation on the Adhesion Activity of Tau Oligomers to Cell Surface by Immunofluorescence Staining

Cal-520 AM was loaded to the NT2-N cells prepared in the above (4-1) by the procedure of the above (4-2), exposed to the tau oligomer sample (10 μl/ml) prepared in the above (1-3) and incubated on ice for 30 minutes. Subsequently, the cells were washed 3 times with DPBS(−) (137 mM of NaCl, 2.68 mM of KCl, 1.47 mM of KH₂PO₄, 8.06 mM of Na₂HPO₄) and fixed with 4% paraformaldehyde at room temperature for 15 minutes. After thorough washing with DPBS(−) (10 minutes×3 times), blocking was done with 1% BSA and 3% donkey serum/DPBS for 30 minutes. Then, the anti-phosphorylated tau antibody (anti-pTau (paired pS409, pS412, pS413), AnaSpec, #AS-55416) (1:1500 dilution) as the primary antibody was reacted at room temperature for 1 hour, and Alexa594-labelled anti-mouse IgG (Jackson Immuno Research, #711-585-152) (1:1500 dilution) as a secondary antibody was reacted at room temperature for 30 minutes, to thereby carry out immunostaining. The stained cells were observed using a fluorescence microscope (BZ-X710, Keyence Corporation).

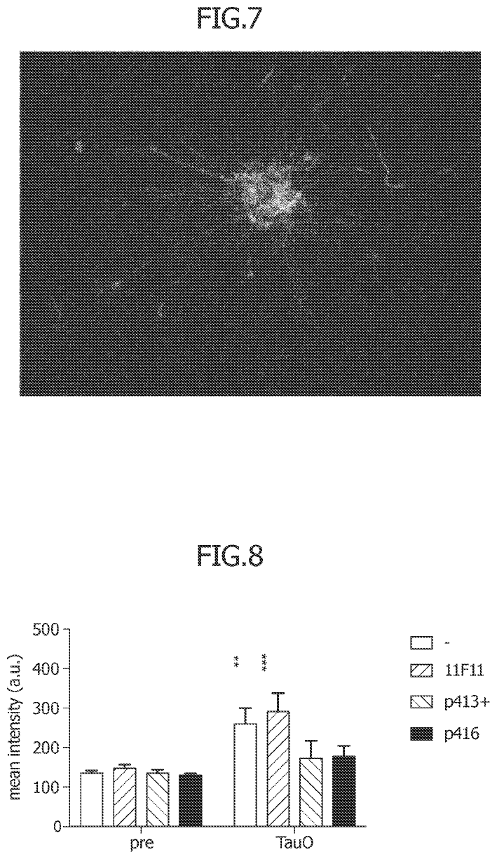

The result is shown in FIG. 7. Since the cells are fixed without membrane permeabilization in the above procedure, the antibodies do not infiltrate into the cells, and the tau oligomers adhered to the cell surface are stained. As a result of the fluorescence microscopic observation, it was revealed that tau oligomers adhered in a patchy pattern to the surface of cell bodies and neurites of the cells. This result showed that the tau oligomer has the adhesion activity to the cell membrane.

### (5-2) Evaluation on the Adhesion Activity of Tau Oligomers to Cell Surface Using a Fluorescence-Labelled HT-Tau Oligomer Sample

The HT-tau oligomer sample prepared in the above (1-4), instead of the tau oligomer sample prepared in the above (1-3), was exposed to the NT2-N cells by the same procedure as in the above (5-1). After thorough washing, fluorescence microscopic observation was carried out. As a result, a patchy pattern of fluorescence was observed on the surface of cell bodies and neurites of the cells (data not shown), similar to the result of the immunofluorescence staining,

<6. Measurement of Uptake of the Tau Oligomer into a Cell>

### (6-1) Preparation of pH Sensor-Labelled HT-Tau Oligomer

0.5 ml of the tau oligomer sample prepared in the above (1-3) (estimated tau concentration: 300 ng/ml) was reacted with 2 μM of HaloTag AcidiFluor ORANGE Ligand (Goryo Chemical, Inc.) at room temperature for 20 minutes, to thereby label the HT-tau oligomer with a pH-sensitive fluorescent probe AcidiFluor ORANGE. Subsequently, the reaction solution was centrifuged at 4° C., 400,000×g for 1 hour. The obtained pellets were resuspended in 0.5 ml of HHBS, and an HT-tau oligomer sample labelled with AcidiFluor ORANGE was obtained (pH sensor-labelled HT-tau oligomer sample).

### (6-2) Measurement of Endocytosis Using the pH Sensor-Labelled HT-Tau Oligomer

The pH sensor-labelled HT-tau oligomer sample, instead of the tau oligomer sample prepared in the above (1-3), was exposed to the NT2-N cells by the same procedure as in the above (5-1). After thorough washing, fluorescence microscopic observation was carried out using IN Cell Analyzer 2200 (GE Healthcare). Furthermore, the NT2-N cells to which the pH sensor-labelled HT-tau oligomer sample was exposed were prepared in the same manner with the exception of simultaneously adding the pH sensor-labelled HT-tau oligomer sample and 10 μl of an anti-phosphorylated tau antibody (anti-pTau (paired pS409, pS412, pS413), AnaSpec, #AS-55416, (1:1500 dilution); and anti-pTau (pS416), GeneTex, #GTX31121 (1:1500 dilution)) or an anti-lectin antibody 11F11 (abcam, ab23461 (1:1500 dilution)) (negative control), and the fluorescence microscopic observation was similarly carried out.

The results are shown in FIG. 8. When the pH sensor-labelled HT-tau oligomer is internalized into a cell by the endocytosis, a fluorescence intensity increases as pH changes. In the figure, the “pre” represents the results of the NT2-N cells before exposed to the tau oligomer sample, and the “TauO” represents the results of the NT2-N cells exposed to the tau oligomer sample. The uptake of tau oligomers into a cell by endocytosis was confirmed in the NT2-N cells exposed to the tau oligomer sample, whereas it was revealed that both of the two anti-tau antibodies inhibited endocytosis. These results suggested that the tau oligomer induces the endocytosis and that an antibody recognizing phosphorylation in the C-terminal region of tau is effective to inhibit the endocytosis. Furthermore, these results are consistent with the verification result on the NMDAR activation ability of the tau oligomer confirmed in the above Item 4.

### <7. Direct Interaction of the Tau Oligomer and NMDAR>

### (7-1) Evaluation on the Direct Interaction of the Tau Oligomer and NMDAR by Pull-Down Assay

Whether or not the tau oligomer and NMDAR directly bind and interact was confirmed by a pull-down assay using the HT-tau oligomer. Magne™ HaloTag beads (Promega Corporation, #G728A) (50 mg) were added to the HT-tau oligomer sample (100 μl) prepared in the above (1-4), and a binding reaction was carried out at 4° C. for 12 hours to thereby immobilize the HT-tau oligomers on the beads. Additionally, brain slice specimens were prepared from tau knockout mice (B6.129X1-Mapt^{tm1Hnd}/J, Jackson Laboratory) by the same procedure as in the above (2-1) and were homogenized in 50-fold volume of the lysis buffer (4 mM of HEPES, 2 mM of EGTA, 0.32 M of sucrose, 1% Protease Inhibitor Cocktail, 1% Phosphatase Inhibitor Cocktail (Nacalai Tesque Inc., #07575-51)), and the lysate was centrifuged at 4° C., 12,000×g for 15 minutes to thereby collect pellets. The obtained pellets were dissolved in a 2% cholic acid or 1% deoxycholate/TBS solution (25 mM of Tris-HCl, 150 mM of NaCl, pH 7.4) and the resultant was used as a membrane protein solution. The membrane protein solution (100 μl) was mixed with the beads on which the HT-tau oligomers were immobilized in 1 ml of 0.1% Triton X-100 solution (50 mM of HEPES, 150 mM of NaCl, pH 7.4) and reacted at 4° C. for 12 hours. Then, the tau oligomer was cleaved and detached from the HaloTag by using TEV protease, to thereby collect the tau oligomer-membrane protein complexes. The obtained tau oligomer-membrane protein complexes were applied to SDS-PAGE electrophoresis, and NMDARs were detected by the western blot. For detection, an anti-NR1 subunit antibody (Merck Millipore, #MAN363) (1:2000 dilution) was used.

The results are shown in FIG. 9. In the figure, the “Input” represents the result of the membrane protein solution before the coimmunoprecipitation (positive control), and the “FT” represents the result of the membrane protein solution after coimmunoprecipitation. The coimmunoprecipitation confirmed a reduced amount of NMDAR in the membrane protein solution. The “TEV” shows the result of the tau oligomer-membrane protein complexes collected from the coimmunoprecipitation. This result revealed that NMDARs had directly bound to the tau oligomer.

### (7-2) Evaluation on the Direct Interaction of the Tau Oligomer and NMDAR by the Far-Western Blot

The membrane protein prepared by the procedure of the above (7-1) was applied to blue native PAGE (NativePAGE™ 3-12% Bis-Tris Protein Gel (Thermo Fisher Scientific, #BN1003BOX); NativePAGE™ Sample Prep Kit (Thermo Fisher Scientific, #BN2008); and NativePAGE™ Running Buffer Kit (Thermo Fisher Scientific, #BN2007) were used) to thereby obtain crude purified NMDAR (complex of a NMDAR4 tetramer and scaffolding proteins such as PSD95, hereinafter simply described as “NMDAR complex”). The NMDAR complex was transferred from the gel after electrophoresis to PVDF membrane (Merck Millipore, #IPVH00010) (transcription buffer: 25 mM of Tris, 192 mM of glycine, 0.1% SDS, 10% Methanol, pH 8.0). The PVDF membrane after transfer was incubated in a solution containing 0.1% of n-Dodecyl-β-D-maltopyranoside (DDM)/50 mM of HEPES, 150 mM of NaCl, pH 7.4) at room temperature for 30 minutes, and the NMDAR complex immobilized on the PVDF membrane was reconstituted. Subsequently, the PVDF membrane was thoroughly washed (wash buffer: 50 mM of HEPES, 150 mM of NaCl, 0.02% DDM, pH 7.4) and was blocked with 5% skim milk/wash buffer. Then, the PVDF membrane was washed and exposed to the HT-tau oligomer sample prepared in the above (1-4) (room temperature, 30 minutes). Subsequently, the membrane was washed, and the tau oligomer adsorbed onto the PVDF membrane was detected by an anti-HaloTag antibody (Promega Corporation, #G928A) (1:1500 dilution) and an HRP-labelled secondary antibody (Jackson Immuno Research, #111-035-144). Additionally, in a replication experiment carried out in parallel, the NMDAR complex was detected by the same procedure without the exposing step to the tau oligomer sample and using an anti-NR1 antibody (Merck Millipore, #MAB363) (1:2000 dilution), instead of the anti-HaloTag antibody.

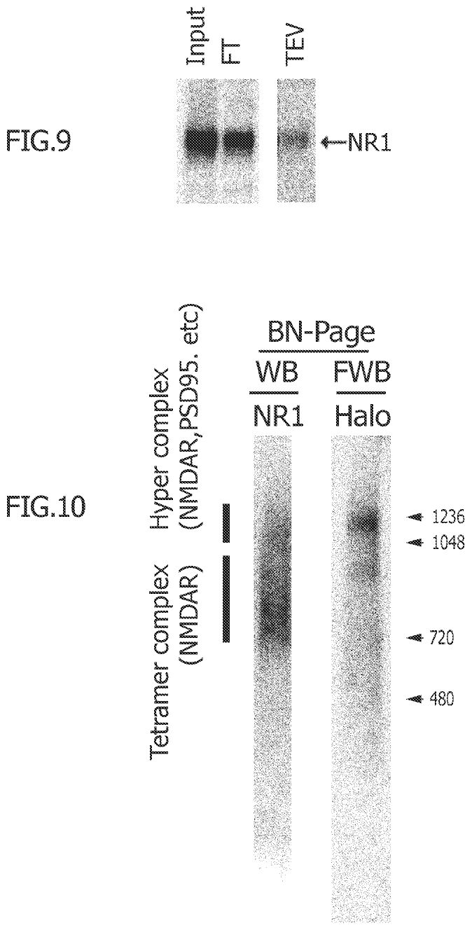

The results are shown in FIG. 10. In the figure, the “WB/NR1” represents the NMDAR complex detected by the western blot, and the “FWB/Halo” represents the HT-tau oligomer detected by the far-western blot. The NMDAR complex was detected around 800 kDa and 1100 kDa, and the HT-tau oligomer was confirmed to have interacted with both of them. These results suggested that the tau oligomer directly interacts with NMDAR with high selectivity.

(7-3) Identification of the Tau Oligomer Directly Interacting with NMDAR

The membrane protein solution prepared by the procedure of the above (7-1) was diluted 16-fold with the above wash buffer and was spotted (1.5 μl/spot) on a nitrocellulose membrane. Then, the membrane was thoroughly dried to thereby obtain a nitrocellulose membrane on which the NMDAR-containing membrane protein was immobilized. On the other hand, a reaction solution before ultracentrifugation to be obtained in the process of preparing the HT-tau oligomer sample of the above (1-4) was fractionated using a gel filtration column (Superdex 200 10/300 GL column, GE Healthcare) (flow rate 0.5 ml/min, fractionated by 1 ml) to thereby obtain tau oligomer fractions in various sizes. Each of the obtained tau oligomer fractions was exposed to the nitrocellulose membrane on which the NMDAR-containing membrane protein was immobilized, to thereby detect the tau oligomers adsorbed onto the nitrocellulose membrane in the same manner as the detection procedure of tau oligomer in the above (7-2).

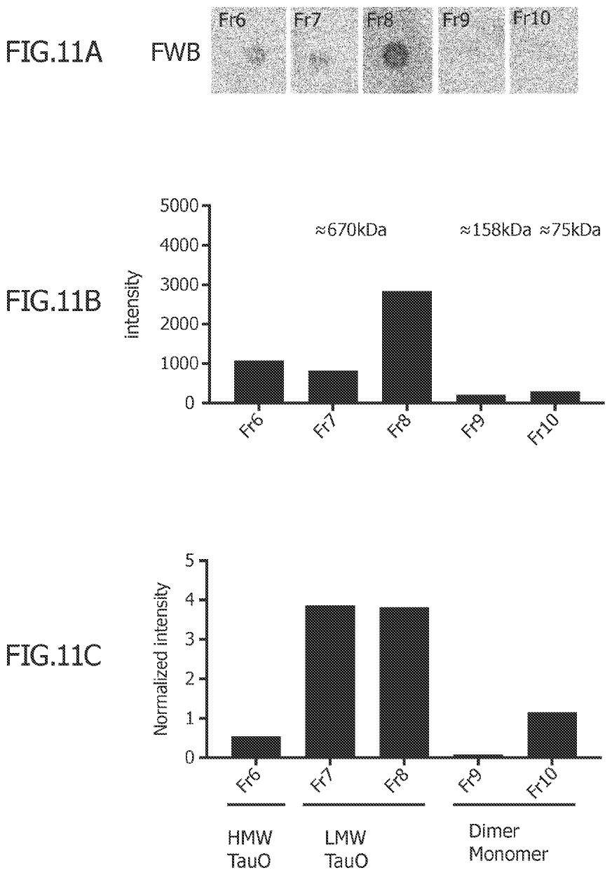

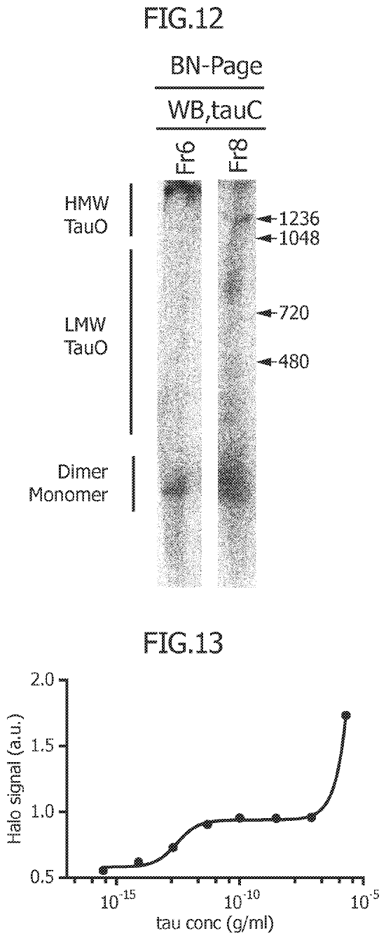

The results are shown in FIGS. 11A to 11C, and 12. FIG. 11A shows the results of the dot blot of the tau oligomer fractions 6 to 10, FIG. 11B shows a graph of the quantified results of FIG. 11A, and FIG. 11C shows a graph of the normalized results of FIG. 11B to the tau oligomer relative frequency of each fraction estimated at an absorbance of 280 nm. This result shows that comparatively low-molecular-weight-tau oligomers (LMW TauO) contained in the fractions 7 and 8 have strong binding activity to NMDAR in comparison with the high-molecular-weight-tau oligomer (HMW TauO) contained in the fraction 6 and the tau monomer and dimer contained in the fractions 9 and 10. Additionally, FIG. 12 shows the results of the blue native PAGE/western blot of the fractions 6 and 8 (primary antibody: TauC (Dako, #A0024) (1:2000 dilution)). Strong signals were confirmed at 13000 kDa or higher for the fraction 6 and near 800 kDa for the fraction 8. The tau oligomer at 800 kDa was estimated to be about a 10-mer, based on the average molecular weight of the tau monomer and the HT-tau monomer.

### (7-4) Evaluation on Direct Interaction of the Tau Oligomer and NMDAR by Membrane Protein-Based Sandwich ELISA

Pellets of the membrane protein obtained from tau knockout brain by the procedure of the above (7-1) were dissolved in 2% cholic acid or 1% deoxycholate/buffer (50 mM of HEPES, 150 mM of NaCl, pH 7.4), and the solution was centrifuged at 4° C., 150,000×g for 30 minutes to thereby collect the supernatant to use as a membrane protein solution. The obtained membrane protein solution was applied to an ELISA plate (Sumitomo Bakelite Co., Ltd., MS-8596F) and incubated at 4° C. for 60 minutes to thereby coat the plate with the membrane protein. After washing with wash solution (0.004 to 0.008% MNG-3, 50 mM of HEPES, pH 7.4), blocking was done by adding 1% FBS-containing wash solution and incubating at 4° C. for 60 minutes. The solvent of the HT-tau oligomer sample prepared in the above (1-4) was replaced with the wash solution using a protein concentrator (10K MWCO, Pierce, 88526) to thereby prepare a tau oligomer solution of 10⁻¹⁶ to 10⁻⁶ g/ml. These solutions were applied to the plate and incubated at 4° C. for 15 to 60 minutes. Then, the plate was washed thoroughly with the wash solution and a 5% skim milk-containing wash solution was added, followed by incubating at room temperature for 30 minutes to thereby block the protein. After washing using the wash solution, an anti-HaloTag antibody/wash solution (1:1000 dilution) was added and incubated at room temperature for 30 minutes. Subsequently, the plate was washed with the wash solution, an HRP-labelled secondary antibody/wash solution (Invitrogen, A27036) (1:5000 dilution) was added and incubated at room temperature for 30 minutes. Then, chromogenic reactions were carried out using an Ultra TMB solution (Thermo Fisher Scientific, 34028) to detect the tau oligomers bound to the membrane protein.

The results are shown in FIG. 13. It was confirmed that the binding of the tau oligomer was detected even when the tau oligomer solution having a low concentration of 10⁻¹¹ g/ml or lower was provided, and the amount of tau oligomer bound increased in a concentration-dependent manner when the tau oligomer solution having 10⁻⁸ g/ml or higher was provided. The binding of the tau oligomer at low concentrations was reduced in the presence of a NMDAR ligand (glutamate and glycine) (data not shown), and the tau oligomers were presumed to be bound to NMDARs.

### (7-5) Evaluation on Direct Interaction of the Tau Oligomer and NMDAR by NMDAR-Based Sandwich ELISA

Antibody solutions recognizing the C-terminal region of NR2a and NR2b subunits of NMDA receptor, (anti-NR2A antibody (BD Biosciences, #612286)/PBS and anti-NR2B antibody (BD Biosciences, #610416)/PBS, each at 1:500 dilution) was applied to an ELISA plate and incubated at 4° C. for 60 minutes to thereby coat the plate with the antibodies. Blocking was done by adding 1% FBS-containing wash solution and incubating at 4° C. for 60 minutes. A membrane protein solution, in which the solvent was replaced with the wash solution by the same procedure as in the above (7-4) using a protein concentrator (100K MWCO, Pierce, 88523), was applied to the plate and incubated at 4° C. for 15 to 60 minutes to thereby immobilize the NMDA receptor on the plate. The tau oligomers bound to the NMDA receptor were detected by the same procedure as in the above (7-4), except that the tau oligomer sample prepared in the above (1-3), instead of the HT-tau oligomer sample, and an anti-tau antibody (TauC), instead of the anti-HaloTag antibody, were used.

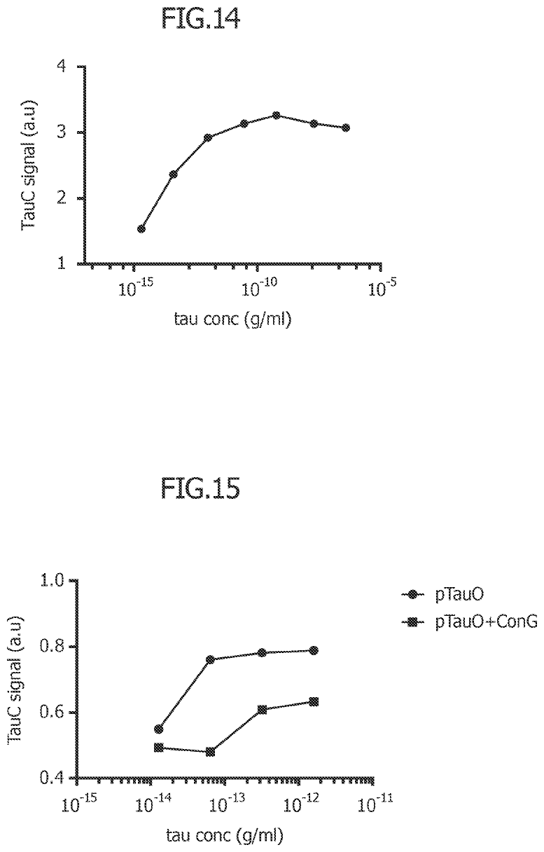

The results are shown in FIG. 14. The binding of the tau oligomer to the NMDA receptor increased in a concentration-dependent manner when the tau oligomer solution of 10⁻¹¹ g/ml or less was applied.

Subsequently, the binding of the tau oligomer to the NMDA receptor was detected in the same manner as above except that conantokin-G (10 μM, Peptide Institute, Inc.), which is a peptide blocking a ligand-binding site of the NMDA receptor, was added to the tau oligomer solution. The results are shown in FIG. 15. In the figure, the “pTauO” represents the result when using the solution of only the tau oligomer and the “pTauO+ConG” represents the result when using the tau oligomer solution added conantokin-G. It was shown that conantokin-G inhibited the binding of the tau oligomer to the NMDA receptor. These results confirmed that the tau oligomer binds to a ligand-binding site of the NMDA receptor.

The above results confirmed that the tau oligomer specifically and directly binds to the NMDA receptor. Furthermore, cerebrospinal fluid samples derived from Alzheimer's disease patient (male, 75 years old) and a healthy old person (male, 71 years old) (obtained from PrecisionMed Inc., 1 sample each) were analyzed by the same procedure as in the above (7-5) and the tau oligomers in the samples were quantified. As a result, it was found that 30 pg/ml of tau oligomers was contained in the sample of the Alzheimer's disease patient, and that 1 pg/ml of tau oligomers was contained in the sample of the healthy old person. These results showed that the quantification of the tau oligomers directly binding to the NMDA receptor enables the diagnose for tauopathy such as Alzheimer's disease.

### (7-6) Isolation and Purification of NMDAR-Binding Tau Oligomer

The tau oligomer sample prepared in the above (1-3) was ultracentrifuged at 4° C., 100,000 rpm for 1 hour, thereby yielding a mixture of various tau oligomers and fibrous polymers as pellets. 100% Formic acid (Nacalai Tesque Inc.) was added to the obtained pellets (final concentration 88%) and the resultant was incubated at 4° C. for 1 hour. The eluted phosphorylated tau monomer was collected by centrifugation at 4° C., 50,000 rpm, lyophilized, and then dissolved in buffer (50 mM of HEPES, pH 7.4). Tau oligomer was prepared by the same procedure as in the above (1-3) except that 0.0025 mM of thioflavin T (Sigma-Aldrich, #T3516-5G) was added, and the re-oligomerization of the tau was confirmed using the incorporation of thioflavin T as an indicator.

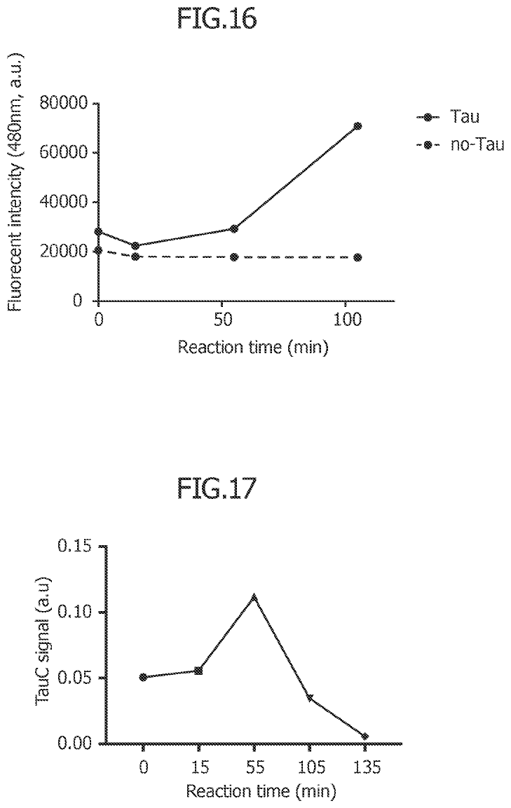

The results are shown in FIG. 16. In the figure, the “Tau” shows the result of the phosphorylated tau monomer+thioflavin T solution, and the “no-Tau” represents the result of the solution of only thioflavin T in the same procedure (negative control). The reconstitution of the tau oligomer was confirmed.

Additionally, the binding of the tau oligomer to the NMDA receptor was detected by the same procedure as in the above (7-4) except that the above phosphorylated tau monomer solution (prepared to be 2×10⁹ g/ml) was used instead of the tau oligomer solution. The results are shown in FIG. 17. In light of the time course of oligomerization shown in FIG. 16, it was confirmed that the peak of binding activity of the tau oligomer was observed immediately before the advanced tau fibril formation. These results suggested the possibility of isolating and purifying the tau oligomers exhibiting strong neurotoxicity using the binding to NMDARs as an indicator.

1. A screening method for an agent for treating or preventing tauopathy, comprising:
(1) a step of contacting an NMDA-type glutamate receptor with a tau oligomer in the presence or absence of a candidate compound, and
(2) a step of evaluating a direct binding of the tau oligomer to the NMDA-type glutamate receptor.

2. The method according to claim 1, wherein the NMDA-type glutamate receptor is isolated from a cell membrane or a liposomal membrane while the NMDA-type glutamate receptor maintains a quaternary structure.

3. The method according to claim 1, wherein the NMDA-type glutamate receptor is contained on a cell membrane or a liposomal membrane while the NMDA-type glutamate receptor maintains a physiological function.

4. The method according to claim 1, wherein the tau oligomer or the NMDA-type glutamate receptor is immobilized on a solid support.

5. The method according to claim 1, wherein the step (2) is carried out by ELISA, a protein array, or a surface plasmon resonance analysis.

6. The method according to claim 3, further comprising (3) a step of measuring a calcium influx through the NMDA-type glutamate receptor into a cell or a liposome.

7. The method according to claim 3, further comprising (4) a step of measuring incorporation of a membrane protein into a cell or a liposome.

8. The method according to claim 1, wherein the tau oligomer consists of 2 to 40 tau proteins.

9. The method according to claim 1, wherein the tau oligomer consists of 3 to 20 tau proteins.

10. The method according to claim 1, wherein the tau oligomer comprises a tau protein as a structural component, the tau protein comprising a phosphorylated amino acid in the C-terminal region downstream of an amino acid corresponding to the 373 position numbered according to the 2N4R isoform.

11. The method according to claim 1, wherein the tau oligomer comprises a tau protein as a structural component, the tau protein comprising in which serine corresponding to the 409, 412, 413 and/or 416 position numbered according to the 2N4R isoform is phosphorylated.

12. The method according to claim 1, wherein the tauopathy is Alzheimer's disease, corticobasal degeneration, progressive supranuclear palsy, Pick's disease, argyrophilic grain dementia, multiple system tauopathy with presenile dementia (MSTD), frontotemporal dementia with parkinsonism linked to chromosome 17 (FTDP-17), dementia with neurofibrillary tangles, diffuse neurofibrillary tangles with calcification (DNTC), white matter tauopathy with globular glial inclusions (WMT-GGI), or frontotemporal lobar degeneration with tau-positive inclusions (FTLD-tau).

13. A test method for tauopathy, comprising:
(1) a step of contacting an NMDA-type glutamate receptor with a sample isolated from a subject, and
(2) a step of quantifying tau oligomers directly binding to the NMDA-type glutamate receptor.

14. The method according to claim 13, wherein the NMDA-type glutamate receptor is isolated from a cell membrane or a liposomal membrane while the NMDA-type glutamate receptor maintains a quaternary structure.

15. The method according to claim 13, wherein the NMDA-type glutamate receptor is contained on a cell membrane or a liposomal membrane while the NMDA-type glutamate receptor maintains a physiological function.

16. The method according to claim 13, wherein the tau oligomer or the NMDA-type glutamate receptor is immobilized on a solid support.

17. The method according to claim 13, wherein the step (2) is carried out by ELISA, a protein array, or a surface plasmon resonance analysis.

18. The method according to claim 15, further comprising (3) a step of measuring a calcium influx through the NMDA-type glutamate receptor into a cell or a liposome.

19. The method according to claim 15, further comprising (4) a step of measuring incorporation of a membrane protein into a cell or a liposome.

20. The method according to claim 13, wherein the tau oligomer consists of 2 to 40 tau proteins.

21. The method according to claim 13, wherein the tau oligomer consists of 3 to 20 tau proteins.

22. The method according to claim 13, wherein the tau oligomer comprises a tau protein as a structural component, the tau protein comprising a phosphorylated amino acid in the C-terminal region downstream of an amino acid corresponding to the 373 position numbered according to the 2N4R isoform.

23. The method according to claim 13, wherein the tau oligomer comprises a tau protein as a structural component, the tau protein comprising in which serine corresponding to the 409, 412, 413 and/or 416 position numbered according to the 2N4R isoform is phosphorylated.

24. The method according to claim 13, wherein the tauopathy is Alzheimer's disease, corticobasal degeneration, progressive supranuclear palsy, Pick's disease, argyrophilic grain dementia, multiple system tauopathy with presenile dementia (MSTD), frontotemporal dementia with parkinsonism linked to chromosome 17 (FTDP-17), dementia with neurofibrillary tangles, diffuse neurofibrillary tangles with calcification (DNTC), white matter tauopathy with globular glial inclusions (WMT-GGI), or frontotemporal lobar degeneration with tau-positive inclusions (FTLD-tau).
